(19) United States
(12) Patent Application Publication
(10) Pub. No.: US 2024/0180471 A1
(43) Pub. Date: Jun. 6, 2024

(54) BIOLOGICAL SIGNAL ANALYSIS METHOD

(71) Applicant: VUNO Inc., Seoul (KR)

(72) Inventors: Woong BAE, Seoul (KR); Yunwon TAE, Anyang-si (KR)

(21) Appl. No.: 18/556,517

(22) PCT Filed: Nov. 9, 2021
(86) PCT No.: PCT/KR2021/016200

§ 371 Date: Oct. 20, 2023

(30) Foreign Application Priority Data
Jun. 2, 2021 (KR) 10-2021-0071364

(51) Int. Cl. A61B 5/327; A61B 5/00; A61B 5/346

(52) U.S. Cl. CPC A61B 5/327; A61B 5/346; A61B 5/7246; A61B 5/7278

(57) ABSTRACT

Disclosed is a bio-signal analysis method performed by computing device according to an embodiment of the present disclosure. The method may include: acquiring at least one lead-wise bio-signal from a plurality of leads: and deriving an analysis value by inputting the at least one acquired lead-wise bio-signal into a neural network model.

### BACKGROUND

### Technical Field

The present disclosure relates to an analysis method of a bio-signal, and more particularly, to a method for analyzing asynchronous bio-signals of various combinations based on deep learning.

### Description of the Related Art

Smart Healthcare is an industrial field that deals with information, devices, systems, and platforms related to personal health and medical care. The smart healthcare aims to provide individuals with appropriate health care methods or customized medical guidance by collecting and analyzing a variety of bio-signals generated from the human body through sensors. Therefore, in a smart healthcare environment, it is one of the important issues how to secure and analyze the bio-signal.

One of the most representative bio-signals used in the smart healthcare environment is an ECG signal. In the smart healthcare environment, the ECG signal is obtained in an asynchronous state through a combination of various leads. In the related art, each combination has established an independent deep learning model to analyze the ECG signal. For example, assuming that an ECG signals through combination of 12 leads is obtained, there may be 4095 combinations of ECG signals. Therefore, if the ECG signals is obtained through the combinations of 12 leads, 4095 deep learning models independent for each combination cannot but be required according to the related art. In other words, in the related art, since the deep learning model corresponding to each combination of leads is individually required, the number of combinations of the leads increases, and the cost of calculation for analysis of the ECG signal is inevitably increased.

U.S. patent application Ser. No. 16/827,812 (published on Nov. 12, 2020; now U.S. Pat. No. 11,571,162) discloses a method for classifying atrial fibrillation by using a single lead ECG.

### BRIEF SUMMARY

### Technical Problem

The present disclosure is contrived in response to the background art, and has been made in an effort to provide a method which analyzes asynchronous bio-signals of various combinations through a single model to derive a desired analysis value even though there are only some of the asynchronous bio-signals of various combinations.

### Technical Solution

In order to implement the object, disclosed is a bio-signal analysis method performed by a computing device including at least one processor according to an embodiment of the present disclosure. The method may include: acquiring at least one lead-wise bio-signal from a plurality of leads; and deriving an analysis value by inputting the acquired at least one lead-wise bio-signal into a neural network model. In this case, the analysis value may be derived by reflecting a correlation between the plurality of leads based on the input at least one lead-wise bio-signal regardless of a combination of lead-wise bio-signals which are enabled to be acquired from the plurality of leads.

In an alternative embodiment, the deriving of the analysis value targeted by the neural network model may include extracting a feature for the acquired at least one lead-wise bio-signal by using the neural network model, encoding positional information of a lead in which the bio-signal is acquired to the extracted feature by using the neural network model, and deriving the analysis value to which the correlation between the plurality of leads is reflected based on the feature encoded with the positional information of the lead by using the neural network model.

In an alternative embodiment, the deriving of the analysis value to which the correlation between the plurality of leads is reflected based on the feature encoded with the positional information of the lead may include performing a self-attention based computation for reflecting the correlation between the plurality of leads based on the feature encoded with the positional information of the lead by using the neural network model, and deriving the analysis value based on a result of the self-attention based computation by using the neural network model.

In an alternative embodiment, the performing of the self-attention based computation for reflecting the correlation between the plurality of leads may include generating a matrix for representing the correlation between the plurality of leads based on the feature encoded with the positional information of the lead by using the neural network model, and deriving the result of the self-attention based computation based on the matrix by using the neural network model.

In an alternative embodiment, the generating of the matrix for representing the correlation between the plurality of leads based on the feature encoded with the positional information of the lead may include generating a query vector, a key vector, and a value vector based on the feature encoded with the positional information of the lead by using the neural network model, and generating a multi-head matrix based on the query vector and the key vector by using the neural network model.

In an alternative embodiment, the deriving of the result of the self-attention based computation based on the matrix may include deriving a weighted sum of the value vector based on the multi-head matrix by using the neural network model.

In an alternative embodiment, the generating of the multi-head matrix based on the query vector and the key vector may include masking a matrix value corresponding to a lead in which the bio-signal is not acquired in the multi-head matrix.

In an alternative embodiment, the masking may be to process the matrix value of the multi-head matrix as 0.

In an alternative embodiment, the neural network model may be pre-trained by randomly masking the matrix generated based on lead-wise bio-signals acquired in all of the plurality of leads and for representing the correlation between the plurality of leads.

In order to implement the object, disclosed is a computer program stored in a computer-readable storage medium according to an embodiment of the present disclosure. The computer program executes the following operations for analyzing a bio-signal when the computer program is executed by one or more processor and the operations may include: an operation of acquiring at least one lead-wise bio-signal from a plurality of leads; and an operation of deriving an analysis value by inputting the acquired at least one lead-wise bio-signal into a neural network model. In this case, the analysis value may be derived by reflecting a correlation between the plurality of leads based on the input at least one lead-wise bio-signal regardless of a combination of lead-wise bio-signals which are enabled to be acquired from the plurality of leads.

In order to implement the object, disclosed is a computing device analyzing a bio-signal according to an embodiment of the present disclosure. The device may include: a processor including at least one core; a memory including program codes executable in the processor; and a network unit acquiring at least one lead-wise bio-signal from a plurality of leads, and the processor may derive an analysis value by inputting the acquired at least one lead-wise bio-signal into a neural network model. In this case, the analysis value may be derived by reflecting a correlation between the plurality of leads based on the input at least one lead-wise bio-signal regardless of a combination of lead-wise bio-signals which are enabled to be acquired from the plurality of leads.

### Advantageous Effects

The present disclosure can provide a method which analyzes asynchronous bio-signals of various combinations through a single model to derive a desired analysis value even though there are only some of the asynchronous bio-signals of various combinations.

### BRIEF DESCRIPTION OF THE SEVERAL VIEWS OF THE DRAWINGS

FIG. 1 is a block diagram of a computing device for analyzing a bio-signal according to an embodiment of the present disclosure.

FIG. 2 is a schematic view illustrating a neural network according to an embodiment of the present disclosure.

FIG. 3 is a block diagram illustrating a bio-signal analysis process by the computing device according to an embodiment of the present disclosure.

FIG. 4 is a conceptual view illustrating a structure of a neural network model according to an embodiment of the present disclosure.

FIG. 5 is a flowchart illustrating a method for analyzing a bio-signal according to an embodiment of the present disclosure.

FIG. 6 is a flowchart illustrating a method for analyzing an ECG signal according to an embodiment of the present disclosure.

FIG. 7 is a schematic view of a computing environment according to an embodiment of the present disclosure.

### DETAILED DESCRIPTION

Hereinafter, various embodiments are described with reference to the drawings. In the present specification, various descriptions are presented for understanding the present disclosure. However, it is obvious that the embodiments may be carried out even without a particular description.

Terms, “component,” “module,” “system,” and the like used in the present specification indicate a computer-related entity, hardware, firmware, software, a combination of software and hardware, or execution of software. For example, a component may be a procedure executed in a processor, a processor, an object, an execution thread, a program, and/or a computer, but is not limited thereto. For example, both an application executed in a computing device and the computing device may be components. One or more components may reside within a processor and/or an execution thread. One component may be localized within one computer. One component may be distributed between two or more computers. Further, the components may be executed by various computer readable medium having various data structures stored therein. For example, components may communicate through local and/or remote processing according to a signal (for example, data transmitted to another system through a network, such as Internet, through data and/or a signal from one component interacting with another component in a local system and a distributed system) having one or more data packets.

A term “or” intends to mean comprehensive “or,” not exclusive “or.” That is, unless otherwise specified or when it is unclear in context, “X uses A or B” intends to mean one of the natural comprehensive substitutions. That is, when X uses A, X uses B, or X uses both A and B, “X uses A or B” may be applied to any one among the cases. Further, a term “and/or” used in the present specification shall be understood to designate and include all of the possible combinations of one or more items among the listed relevant items.

A term “include” and/or “including” shall be understood as meaning that a corresponding characteristic and/or a constituent element exists. Further, a term “include” and/or “including” means that a corresponding characteristic and/or a constituent element exists, but it shall be understood that the existence or an addition of one or more other characteristics, constituent elements, and/or a group thereof is not excluded. Further, unless otherwise specified or when it is unclear that a single form is indicated in context, the singular shall be construed to generally mean “one or more” in the present specification and the claims.

The term “at least one of A or B” should be interpreted to mean “a case including only A,” “a case including only B,” and “a case in which A and B are combined.”

Those skilled in the art need to recognize that various illustrative logical blocks, configurations, modules, circuits, means, logic, and algorithm steps described in connection with the embodiments disclosed herein may be additionally implemented as electronic hardware, computer software, or combinations of both sides. To clearly illustrate the interchangeability of hardware and software, various illustrative components, blocks, constitutions, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented embodiments is provided so that those skilled in the art of the present disclosure use or implement the present disclosure. Various modifications to the embodiments will be apparent to those skilled in the art. Generic principles defined herein may be applied to other embodiments without departing from the scope of the present disclosure. Therefore, the present disclosure is not limited to the embodiments presented herein. The present disclosure should be analyzed within the widest range which is coherent with the principles and new features presented herein.

In the present disclosure, a neural network, an artificial neural network, and a network function may be exchangeably used in some cases.

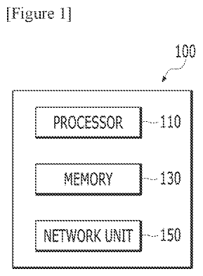

FIG. 1 is a block diagram of a computing device for analyzing a bio-signal according to an embodiment of the present disclosure.

A configuration of the computing device 100 illustrated in FIG. 1 is only an example simplified and illustrated. In an exemplary embodiment of the present disclosure, the computing device 100 may include other components for performing a computing environment of the computing device 100, and only some of the disclosed components may constitute the computing device 100.

The computing device 100 may include a processor 110, a memory 130, and a network unit 150.

According to an exemplary embodiment, the processor 110 may be constituted by one or more cores, and include processors for data analysis and deep learning, such as a central processing unit (CPU), a general purpose graphics processing unit (GPGPU), a tensor processing unit (TPU), etc., of the computing device. The processor 110 may read a computer program stored in the memory 130 and process data for machine learning according to an exemplary embodiment of the present disclosure. According to an exemplary embodiment of the present disclosure, the processor 110 may perform an operation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, the GPGPU, and the TPU of the processor 110 may process learning of the network function. For example, the CPU and the GPGPU may process the learning of the network function and data classification using the network function jointly. In addition, in an exemplary embodiment of the present disclosure, the learning of the network function and the data classification using the network function may be processed by using processors of a plurality of computing devices together. In addition, the computer program performed by the computing device according to an exemplary embodiment of the present disclosure may be a CPU, GPGPU, or TPU executable program.

According to an embodiment of the present disclosure, the processor 110 may train a neural network model for analyzing bio-signals based on bio-signals acquired from a plurality of leads. The processor 110 may train a neural network model to derive an analysis value to which a correlation between the plurality of leads is reflected based on lead-wise bio-signals corresponding to the plurality of leads, respectively. For example, the processor 110 may input bio-signals acquired from 12 leads into one neural network model. The processor 110 may train the neural network model so as for the neural network model receiving the bio-signals acquired from 12 leads to derive a prediction value of a cardiovascular disease such as myocardial infarction (MI) to which a correlation between 12 leads is reflected. When one neural network model is trained to derive the prediction value of the cardiovascular disease by reflecting the correlation between 12 leads as described above, the neural network model may accurately derive the prediction value of the cardiovascular disease even though all bio-signals are not acquired from 12 leads, but only some of the bio-signals are acquired. That is, without individually constructing models by considering the number of cases of all combinations which are enabled to be acquired from 12 leads, the processor 110 may perform accurate analysis with a single model constructed through the training to which the correlation between the leads is reflected as described above.

The processor 110 may derive the analysis value to which the correlation between the asynchronous bio-signals of various combinations is reflected by using a pre-trained neural network model as described above. The processor 110 may derive an analysis result for a subject based on at least one lead-wise bio-signal acquired from the plurality of leads through the pre-trained neural network model. That is, the processor 110 may accurately derive an analysis value targeted by the neural network model even though any combination of lead-wise bio-signal is acquired from each of the plurality of leads used for training the neural network model through the pre-trained neural network model as described above. In other words, the processor 110 may accurately derive an analysis value desired by a user by reflecting the correlation between the leas from which the bio-signal is acquired through one pre-trained neural network model even though any combination of bio-signal is input. For example, the processor 110 may input some combinations of the bio-signals acquired from 12 leads into the pre-trained neural network model. The processor 110 may derive the prediction value of the cardiovascular disease such as the myocardial infarction based on some combinations of the lead-wise bio-signals acquired from 12 leads through the pre-trained neural network model. That is, the processor 110 may accurately derive the prediction value of the cardiovascular disease by reflecting the correlation between 12 leads through one neural network model even in a state in which not all combinations but only some combinations of the lead-wise bio-signals acquired from 12 leads are secured. Further, the processor 110 uses a single model constructed through the training to which the correlation between the leads is reflected to weight-lighten a computing source and remarkably reduce cost required for an operation or a computation of the computing source.

According to an embodiment of the present disclosure, the memory 130 may store any type of information generated or determined by the processor 110 or any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to the exemplary embodiment of the present disclosure may use a predetermined form of a publicly known wired/wireless communication system.

The network unit 150 may receive an ECG signal from a signal measurement system. In this case, the signal measurement system may be understood as a system including all devices capable of measuring, storing, and processing the ECG signal. For example, the signal measurement system may include a portable ECG measuring device including a lead contactable to a body of a subject, a database server that may be linked with the portable ECG measuring device, and the like. The network unit 150 may acquire at least one lead-wise bio-signal measured through a plurality of leads having two or more various combinations through communication with the portable ECG measurement device. The network unit 150 may receive a bio-signal previously measured by the portable ECG measuring device and stored in the database server through communication with the database server. The disclosure is just one example, so the network unit 150 may acquire the bio-signal through various paths or schemes within a category which may be understood by those skilled in the art.

In addition, the network unit 150 may transmit and receive information processed by the processor 110, a user interface, and the like through communication with other terminals. For example, the network unit 150 may provide the user interface generated by the processor 110 to a client (e.g., a user terminal). In addition, the network unit 150 may receive an external input of a user applied to a client and transfer the external input to the processor 110. In this case, the processor 110 may process operations such as outputting, correcting, changing, adding, and the like of information provided through the user interface based on the external input of the user received from the network unit 150.

Meanwhile, according to an embodiment of the present disclosure, the computing device 100 may include a server as a computing system that transmits and receives information through communication with the client. In this case, the client may be any type of terminal which may access the server. For example, the computing device 100 as the server may receive the ECG signal from the signal measurement system, predict a cardiovascular disease, and provide a user interface including a predicted result to the user terminal. At this time, the user terminal may output the user interface received from the computing device 100 as the server, and receive or process information through interaction with the user.

The user terminal may display the user interface provided to provide analysis information (e.g., prediction information of the cardiovascular disease) of the ECG signal delivered from the computing device 100 as the server. Although not separately illustrated, the user terminal may include a network unit receiving the user interface from the computing device 100, a processor including at least one core, a memory, an output unit providing the user interface, and input unit receiving the external input applied from the user.

In an additional embodiment, the computing device 100 may also include any type of terminal that receives data resources generated by an arbitrary server and performs additional information processing.

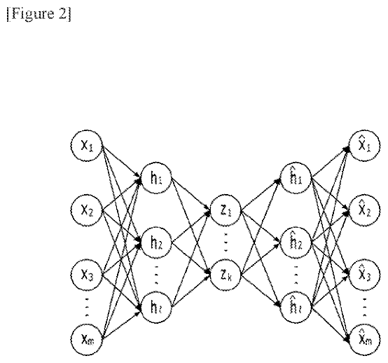

FIG. 2 is a schematic view illustrating a neural network according to an embodiment of the present disclosure.

The neural network model according to an embodiment of the present disclosure may include a neural network for extraction of a feature of the bio-signal, generation of a matrix representing the correlation between leads, etc.

Throughout the present specification, the meanings of a calculation model, a nerve network, the network function, and the neural network may be interchangeably used. The neural network may be formed of a set of interconnected calculation units which are generally referred to as “nodes.” The “nodes” may also be called “neurons.” The neural network consists of one or more nodes. The nodes (or neurons) configuring the neural network may be interconnected by one or more links.

In the neural network, one or more nodes connected through the links may relatively form a relationship of an input node and an output node. The concept of the input node is relative to the concept of the output node, and a predetermined node having an output node relationship with respect to one node may have an input node relationship in a relationship with another node, and a reverse relationship is also available. As described above, the relationship between the input node and the output node may be generated based on the link. One or more output nodes may be connected to one input node through a link, and a reverse case may also be valid.

In the relationship between an input node and an output node connected through one link, a value of the output node data may be determined based on data input to the input node. Herein, a link connecting the input node and the output node may have a weight. The weight is variable, and in order for the neural network to perform a desired function, the weight may be varied by a user or an algorithm. For example, when one or more input nodes are connected to one output node by links, respectively, a value of the output node may be determined based on values input to the input nodes connected to the output node and weights set in the link corresponding to each of the input nodes.

As described above, in the neural network, one or more nodes are connected with each other through one or more links to form a relationship of an input node and an output node in the neural network. A characteristic of the neural network may be determined according to the number of nodes and links in the neural network, a correlation between the nodes and the links, and a value of the weight assigned to each of the links. For example, when there are two neural networks in which the numbers of nodes and links are the same and the weight values between the links are different, the two neural networks may be recognized to be different from each other.

The neural network may consist of a set of one or more nodes. A subset of the nodes configuring the neural network may form a layer. Some of the nodes configuring the neural network may form one layer on the basis of distances from an initial input node. For example, a set of nodes having a distance of n from an initial input node may form n layers. The distance from the initial input node may be defined by the minimum number of links, which need to be passed to reach a corresponding node from the initial input node. However, the definition of the layer is arbitrary for the description, and a degree of the layer in the neural network may be defined by a different method from the foregoing method. For example, the layers of the nodes may be defined by a distance from a final output node.

The initial input node may mean one or more nodes to which data is directly input without passing through a link in a relationship with other nodes among the nodes in the neural network. Otherwise, the initial input node may mean nodes which do not have other input nodes connected through the links in a relationship between the nodes based on the link in the neural network. Similarly, the final output node may mean one or more nodes that do not have an output node in a relationship with other nodes among the nodes in the neural network. Further, the hidden node may mean nodes configuring the neural network, not the initial input node and the final output node.

In the neural network according to the embodiment of the present disclosure, the number of nodes of the input layer may be the same as the number of nodes of the output layer, and the neural network may be in the form that the number of nodes decreases and then increases again from the input layer to the hidden layer. Further, in the neural network according to another embodiment of the present disclosure, the number of nodes of the input layer may be smaller than the number of nodes of the output layer, and the neural network may be in the form that the number of nodes decreases from the input layer to the hidden layer. Further, in the neural network according to another embodiment of the present disclosure, the number of nodes of the input layer may be larger than the number of nodes of the output layer, and the neural network may be in the form that the number of nodes increases from the input layer to the hidden layer. The neural network according to another embodiment of the present disclosure may be the neural network in the form in which the foregoing neural networks are combined.

A deep neural network (DNN) may mean the neural network including a plurality of hidden layers, in addition to an input layer and an output layer. When the DNN is used, it is possible to recognize a latent structure of data. That is, it is possible to recognize latent structures of photos, texts, videos, voice, and music (for example, what objects are in the photos, what the content and emotions of the texts are, and what the content and emotions of the voice are). The DNN may include a convolutional neural network (CNN), a recurrent neural network (RNN), an auto encoder, Generative Adversarial Networks (GAN), a Long Short-Term Memory (LSTM), a transformer, a restricted Boltzmann machine (RBM), a deep belief network (DBN), a Q network, a U network, a Siamese network, a Generative Adversarial Network (GAN), and the like. The foregoing description of the deep neural network is merely illustrative, and the present disclosure is not limited thereto.

In the embodiment of the present disclosure, the network function may include an auto encoder. The auto encoder may be one type of artificial neural network for outputting output data similar to input data. The auto encoder may include at least one hidden layer, and the odd-numbered hidden layers may be disposed between the input/output layers. The number of nodes of each layer may decrease from the number of nodes of the input layer to an intermediate layer called a bottleneck layer (encoding), and then be expanded symmetrically with the decrease from the bottleneck layer to the output layer (symmetric with the input layer). The auto encoder may perform a nonlinear dimension reduction. The number of input layers and the number of output layers may correspond to the dimensions after preprocessing of the input data. In the auto encoder structure, the number of nodes of the hidden layer included in the encoder decreases as a distance from the input layer increases. When the number of nodes of the bottleneck layer (the layer having the smallest number of nodes located between the encoder and the decoder) is too small, the sufficient amount of information may not be transmitted, so that the number of nodes of the bottleneck layer may be maintained in a specific number or more (for example, a half or more of the number of nodes of the input layer and the like).

The neural network may be trained by at least one scheme of supervised learning, unsupervised learning, semi-supervised learning, and reinforcement learning. The training of the neural network may be a process of applying knowledge for the neural network to perform a specific operation to the neural network.

The neural network may be trained in a direction of minimizing an error of an output. In the training of the neural network, training data is repeatedly input to the neural network and an error of an output of the neural network for the training data and a target is calculated, and the error of the neural network is back-propagated in a direction from an output layer to an input layer of the neural network in order to decrease the error, and a weight of each node of the neural network is updated. In the case of the supervised learning, training data labelled with a correct answer (that is, labelled training data) is used, in each training data, and in the case of the unsupervised learning, a correct answer may not be labelled to each training data. That is, for example, the training data in the supervised learning for data classification may be data, in which category is labelled to each of the training data. The labelled training data is input to the neural network and the output (category) of the neural network is compared with the label of the training data to calculate an error. For another example, in the case of the unsupervised learning related to the data classification, training data that is the input is compared with an output of the neural network, so that an error may be calculated. The calculated error is back-propagated in a reverse direction (that is, the direction from the output layer to the input layer) in the neural network, and a connection weight of each of the nodes of the layers of the neural network may be updated according to the backpropagation. A change amount of the updated connection weight of each node may be determined according to a learning rate. The calculation of the neural network for the input data and the backpropagation of the error may configure a learning epoch. The learning rate is differently applicable according to the number of times of repetition of the learning epoch of the neural network. For example, at the initial stage of the learning of the neural network, a high learning rate is used to make the neural network rapidly secure performance of a predetermined level and improve efficiency, and at the latter stage of the learning, a low learning rate is used to improve accuracy.

In the training of the neural network, the training data may be generally a subset of actual data (that is, data to be processed by using the trained neural network), and thus an error for the training data is decreased, but there may exist a learning epoch, in which an error for the actual data is increased. Overfitting is a phenomenon, in which the neural network excessively learns training data, so that an error for actual data is increased. For example, a phenomenon, in which the neural network learning a cat while seeing a yellow cat cannot recognize cats, other than a yellow cat, as cats, is a sort of overfitting. Overfitting may act as a reason of increasing an error of a machine learning algorithm. In order to prevent overfitting, various optimizing methods may be used. In order to prevent overfitting, a method of increasing training data, a regularization method, a dropout method of inactivating a part of nodes of the network during the training process, a method using a bath normalization layer, and the like may be applied.

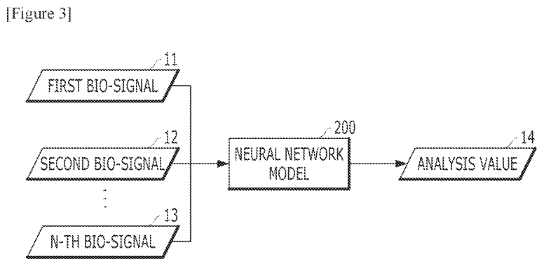

FIG. 3 is a block diagram illustrating a bio-signal analysis process by the computing device according to an embodiment of the present disclosure.

Referring to FIG. 3 the processor 110 of the computing device 100 according to an embodiment of the present disclosure may derive an analysis value for at least one bio-signal which may be acquired as various combinations from a plurality of leads by using a pre-trained neural network model 200. The processor 110 inputs at least one lead-wise bio-signal acquired from the plurality of leads into the neural network model 200 derive an analysis value to which the correlation between the plurality of leads is reflected. That is, the processor 110 may accurately derive an analysis value targeted by the neural network model 200 with respect to all combinations of the bio-signals which may be acquired from the plurality of leads through one neural network model 200 reflecting the correlation between the plurality of leads.

For example, the processor 110 may input N (N is a natural number) lead-wise bio-signals 11, 12, and 13 into the neural network model 200. In this case, N may depend on the combination of the plurality of leads acquiring the bio-signals. That is, the processor 110 may input N lead-wise bio-signals 11, 12, and 13 corresponding to all or some of the plurality of leads into the neural network model 200. The neural network model 200 receives a first bio-signal 11, a second bio-signal 12, and an Nth bio-signal 13 to derive an analysis value 14 for predicting a cardiovascular disease of a subject for which bio-signal is measured. In this case, the analysis value 14 may be a value to which a correlation is reflected for locations, types, etc., of all leads used for acquiring the bio-signals. Therefore, even when the bio-signals are acquired by using only some of all leads for measuring the bio-signals, the neural network model 200 analyzes the lead-wise bio-signals corresponding to some of all leads by reflecting the correlation between all leads to derive the analysis value for predicting the cardiovascular disease of the subject.

Specifically, the processor 110 may extract a feature for at least one lead-wise bio signal acquired from the plurality of leads through the neural network model 200. In this case, the neural network model 200 may be pre-trained based on all lead-wise bio-signals acquired from the plurality of leads in order to extract the feature. For example, when all N bio-signals corresponding to N leads are acquired, the processor 110 may extract a feature of each of N bio-signals through the neural network model 200. Further, even when N bio-signals 11, 12, and 13 corresponding to some combinations of M leads (M is a natural number larger than N) are acquired, the processor 110 may extract the feature of each of N bio-signals through the neural network model 200.

The processor 110 may encode positional information of a lead in which the bio-signal is acquired to the feature of the bio-signal by using the neural network model 200. Since the feature of the lead-wise bio-signal extracted through the neural network model 200 as described above indicates a unique feature of the bio-signal regardless of the lead, the feature does not reflect information related to the lead representing a source of the bio-signal. Therefore, the processor 110 encodes the positional information of the lead in which the bio-signal is acquired to the feature of the lead-wise bio-signal to reflect the information related to the lead to the unique feature of the lead-wise bio-signal. The encoding may be appreciated as a prior task which may reflect the correlation between the plurality of leads to a final analysis value of the neural network model 200. That is, in order for the neural network model 200 to accurately perform the analysis even though any lead-wise signal is input by determining the correlation between the plurality of leads, the processor 110 may encode the positional information of the lead in which the bio-signal is acquired to the feature of the lead-wise bio signal.

The processor 110 may derive the analysis value to which the correlation between the plurality of leads is reflected based on the feature encoded with the positional information of the lead by using the neural network model 200. In order to derive the analysis value for the bio-signal, the processor 110 may perform a computation for generating the correlation between the plurality of leads based on the feature of the bio-signal encoded with the positional information of the lead by using the neural network model 200. The processor 110 may derive the analysis value of the bio-signal to which the correlation between the plurality of leads is reflected based on a result of the computation by using the neural network model 200. For example, the neural network model 200 may perform a self-attention based computation for reflecting the correlation between the plurality of leads by receiving the feature encoded with the positional information of the lead. In this case, the neural network model 200 may include a neural network of a structure such as a transformer for performing the self-attention based computation. The neural network model 200 may define a correlation which is based on positions of leads in the feature of the lead-wise bio-signal through the self-attention based computation. The neural network model 200 may derive an analysis value for predicting the cardiovascular disease based on a result of the self-attention based computation. The correlation between the plurality of leads is reflected to derive the final analysis value through the self-attention based computation, so the neural network model 200 may perform the accurate analysis even though any combination of bio-signal is input from the plurality of leads (even though only bio-signals corresponding to not all but some of the plurality of leads are input). The self-attention based computation is just one example, so various computations for reflecting the correlation between the plurality of leads may be applied within category which may be understood by those skilled in the art.

Meanwhile, the processor 110 may mask information on a lead in which the bio-signal is not acquired in a computation process for deriving the feature. The processor 110 may derive the analysis value by inputting all bio-signals corresponding to the plurality of leads, respectively into to the neural network model 200, but may derive the analysis value by inputting the bio-signals corresponding to some combinations of the plurality of leads into the neural network model 200 through the computation as described above. When the bio-signals corresponding to some combinations of the plurality of leads are used for the analysis, the processor 110 may mask information on the lead in which the bio-signal is not acquired during the computation process of the neural network model 200 in order to process the information on the lead in which the bio-signal is not acquired. In other words, the processor 110 indicates and generates the information on the lead in which the bio-signal is not acquired through masking to effectively perform the computation for reflecting the correlation between the plurality of leads and generate the analysis value.

For example, when bio-signals are acquired from 3 leads, respectively among 12 leads, the processor 110 may extract features of lead-wise bio-signals acquired from 3 leads through the neural network model 200. The processor 110 may encode positional information of 3 respective leads to corresponding features through the neural network model 200. The processor 110 may perform the computation for generating the correlation between the plurality of leads based on the feature of the bio-signal encoded with the positional information of each of 3 leads by using the neural network model 200. In this case, the processor 110 masks information on 9 remaining leads in which the bio-signal is not acquired during the computation process to generate a computation result value for deriving the final analysis value. The processor 110 may derive an analysis value for predicting the cardiovascular disease of the subject based on the computation result value including the masked information through the neural network model 200.

The masking may be randomly performed with respect to all bio-signals acquired from the plurality of leads in the process of training the neural network model 200. All lead-wise bio-signals acquired in all of the plurality of leads are used for an input of the neural network model 200 in the process of training the neural network model 200. Therefore, the processor 110 may perform a kind of random sampling of randomly masking the lead-wise bio-signal acquired in all of the plurality of leads in the process of training the neural network model 200.

For example, when 12 lead-wise bio-signals are acquired from 12 leads and used for training the neural network model 200, the processor 110 may randomly mask some of the 12 lead-wise bio-signals in the computation process of the neural network model 200. A condition of the random sampling such as the number of or types of leads to be masked may vary every learning epoch. The condition of the random sampling such as the number of or types of leads to be masked may vary as predetermined by the user by considering an analysis domain.

When the above description is considered, the neural network model 200 according to an embodiment of the present disclosure reflects the correlation between the plurality of leads to the analysis process of the bio-signal to derive a targeted analysis value regardless of a combination of the lead-wise bio-signals which may be acquired from the plurality of leads. In other words, even though any number and combination of lead-wise bio-signals are input from all or some of the plurality of leads, the neural network model 200 may derive an analysis value (i.e., the analysis value targeted by the neural network model 200) desired by the user through the computation for reflecting the correlation between the plurality of leads.

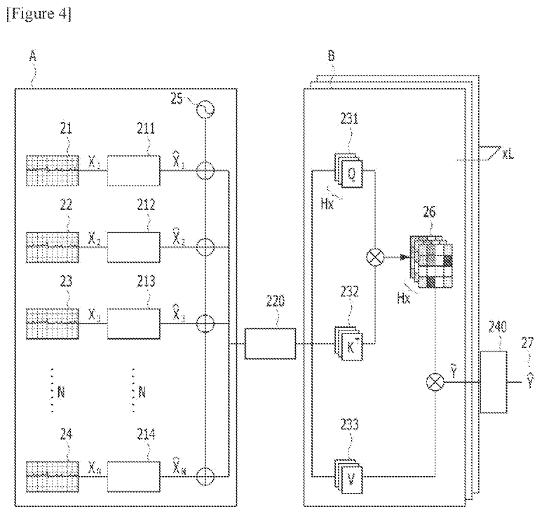

FIG. 4 is a conceptual view illustrating a structure of a neural network model according to an embodiment of the present disclosure.

Referring to FIG. 4, the neural network model according to an embodiment of the present disclosure may include a first neural network A for generating a feature of a bio-signal, a second neural network B performing a self-attention computation for defining a correlation between a plurality of leads, and a third neural network 240 deriving a final analysis value based on a computation result of the second neural network B. Further, the neural network model according to an embodiment of the present disclosure may selectively further include a fourth neural network 220 for concatenating features extracted through the first neural network A.

The first neural network A may include a first encoder for extracting a feature for at least one lead-wise bio-signal acquired from the plurality of leads and a second encoder for including positional information of the lead in each feature extracted through the first encoder. For example, the first neural network A may include N first encoders 211, 212, 213, and 214 and N second encoders corresponding to N leads, respectively. When N bio-signals 21, 22, 23, and 24 are acquired for each lead, N first encoders 211, 212, 213, and 214 for each lead may extract features X from N bio-signals 21, 22, 23, and 24, respectively. In this case, the first encoders 211, 212, 213, and 214 may also be 1-dimensional ResNet based neural networks that share a weight with each other. However, this is just one example, and the type of first encoder is not limited thereto and may be variously configured within category which may be understood by those skilled in the art. When the features \hat{X} of the lead-wise bio-signals are extracted through the first encoders 211, 212, 213, and 214, N second encoders may encode lead-wise positional information 25 that matches each of the bio-signal with respect to each of the features \hat{X} of N lead-wise bio-signals. In this case, each of the second encoders may include a fully connected neural network and may include an encoder that includes the lead-wise positional information in the feature \hat{X} as a specific numerical code.

The second neural network B may perform a self-attention based computation for representing a correlation of all leads based on the feature of the lead-wise bio-signal encoded through the first neural network A. In this case, the second neural network B may include at least one neural network for performing the self-attention based computation. As the number of neural networks included in the second neural network B increases, a depth of the second neural network B may increase. For example, the second neural network B included in L (L is a natural number) multiple neural networks may generate a matrix for representing a correlation between N leads based on the feature \hat{X} of the lead-wise bio-signal encoded with the positional information of the lead. In this case, the features \hat{X} may also be values mutually concatenated through the fourth neural network 220. The second neural network B may generate a query vector 231, a key vector 232, and a value vector 233 based on the feature \hat{X} of the bio-signal encoded with the positional information of the lead. The second neural network B projects the feature \hat{X} onto a vector space in the form of fully connected neural network based query Q, key K, and value V to generate vectors 231, 232, and 233 for generating the matrix representing the correlation between N leads. The second neural network B may generate a multi-head matrix 26 based on the query vector 231 and the key vector 232. The multi-head matrix 26 may be appreciated as a self-attention based matrix expressing a correlation based on positions between N leads. Here, multi-head may be appreciated as a term that encompasses a single head or a plurality of heads. Specifically, the second neural network B may generate the query vector 231, the key vector 232, and the value vector 233 through H (H is the natural number) multi-heads. The second neural network B may generate H multi-head matrices 26 based on the query vector 231 and the key vector 232 generated through H multi-heads. When it is assumed that two heads (i.e., H is 2) are used to generate the multi-head matrix, one head may be used for determining the correlation between the leads in a single lead criterion. The remaining head may be used for determining a correlation between other leads based on a combination of two leads. That is, the second neural network B may generate one self-attention based matrix based on the features \hat{X} extracted from N lead-wise bio-signals, and extend the self-attention based matrix to H self-attention based matrices through H multi-heads.

h-th (h is a natural number smaller than H) multi-head matrix among H multi-head matrices 26 may be expressed as in [Equation 1] below.

A^{h}=softmax[(Q^{h})(K^{h})^{T}],Q^{h}∈ [symbol] ^{N×D},K^{h}∈ [symbol] ^{N×D},h∈{1, . . . ,H} Equation 1

Where, A^{h} may represent an h-th self-attention based matrix, Q^{h} may represent a query vector corresponding to an h-th head, K^{h} may represent a key vector corresponding to the h-th head, N may represent the number of leads, D may represent a dimension of features, and H may be appreciated as the number of heads. Referring to [Equation 1], it may be appreciated that the self-attention based multi-head matrix is generated through a computation of a softmax function based on the query vector and the key vector. However, [Equation 1] is just one example for generating the self-attention based matrix, so various computation schemes for generating the self-attention based matrix may be applied within the category which may be appreciated by those skilled in the art.

Meanwhile, the second neural network B may mask a matrix value corresponding to a lead in which the bio-signal is not acquired among matrix values of the matrix in the process of generating the multi-head matrix 26. In this case, the masking may be appreciated as processing the matrix value of the matrix as 0. For example, when the bio-signal is not acquired in some leads among N leads, the second neural network B may define all matrix values of the matrix through the mask of processing the matrix value of the lead in which the bio-signal is not acquired in the matrix as 0. That is, even though there is the lead in which the bio-signal is not acquired among N leads, the second neural network B processes the matrix value corresponding to the lead in which the bio-signal is not acquired among the matrix values of the matrix as 0 to generate the self-attention based multi-head matrix 26. In other words, in order to stably and accurately derive the analysis value for the bio-signal even though the bio-signals for some leads are not acquired, the second neural network B may generate the multi-head matrix 26 through the masking processing.

The second neural network B may derive a result of a self-attention based computation based on H multi-head matrices 26 described above. The second neural network B may derive a weighted sum of the value vector 233 based on H multi-head matrices 26. That is, the second neural network B may generate the weighted sum of the value vector 233 as the result of the self-attention based computation by using the multi-head matrix 26 as a weight for calculating the sum of the value vector 233. For example, the weighted sum of the value vector using the h-th multi-head matrix may be expressed as in [Equation 2] below.

$${\tilde{Y}}_{h} = {\sum }_{N} ( {A}^{h} ) ( {V}^{h} ) , {A}^{h} ∈ {ℝ}^{N \times N} , {V}^{h} ∈ {ℝ}^{N \times D} \qquad Equation 2$$

Where, \tilde{Y}ₕ may represent a weighted sum of the value vector using the h-th multi-head matrix, A^{h} may represent the h-th self-attention based matrix, V^{h} may represent the value vector corresponding to the h-th head, N may represent the number of leads, and D may be appreciated as the dimension of the features. Referring to [Equation 2], the result of the self-attention based computation may be appreciated as being a result of computing the sum of the value vector using the self-attention based matrix as the weight. However, [Equation 2] is just one example representing the result of the self-attention based computation using the matrix, so various computation schemes for deriving the result of the self-attention based computation may be applied within the category which may be appreciated by those skilled in the art.

The third neural network 240 may derive a targeted analysis value Ŷ based on the result of the self-attention based computation derived through the second neural network B. The third neural network 240 may derive an analysis result value Ŷ for the bio-signal by receiving the result of the self-attention based computation which is the output of the second neural network B. For example, the third neural network 240 may generate analysis information for the cardiovascular disease of the subject based on the weighted sum of the value vector 233 calculated based on the matrix 26. In this case, the third neural network 240 may be a fully connected neural network that outputs an analysis value for determining whether the subject suffers from the cardiovascular disease or predicting a probability that the cardiovascular disease will occur. However, the cardiovascular disease is just one example related to a health condition or disease of a person. Therefore, the health condition or the type of disease of the person which may be determined or predicted through analysis for the bio-signal is not limited, but may be applied to the present disclosure.

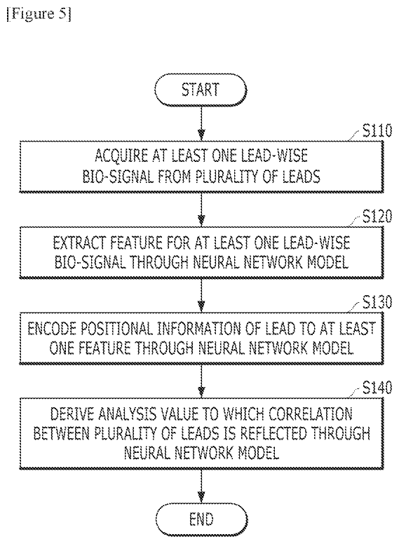

FIG. 5 is a flowchart illustrating a method for analyzing a bio-signal according to an embodiment of the present disclosure.

Referring to FIG. 5, in step S110, a computing device 100 according to an embodiment of the present disclosure may acquire at least one lead-wise bio-signal from a plurality of leads. For example, the computing device 100 may receive a lead-wise bio-signal measured through a plurality of leads provided in a portable measurement equipment such as a smart watch through communication with the portable measurement equipment. The computing device 100 may receive all lead-wise bio-signals measured through all leads provided in the portable measurement equipment. The computing device 100 may receive various combinations of asynchronous bio-signals which may vary depending on a measurement environment, a condition, or a measurement scheme of the lead.

In step S120, the computing device 100 may extract a feature for at least one lead-wise bio-signal by using a neural network model. For example, the computing device 100 may extract the feature by inputting the lead-wise bio-signal acquired through step S110 into the neural network model. In this case, the neural network model may be pre-trained based on all bio-signals acquired according to the number of plurality of leads provided in the portable measurement equipment.

In step S130, the computing device 100 may encode positional information of a lead corresponding to each bio-signal to the feature for at least one lead-wise bio-signal through the neural network model. The computing device 100 may encode positional information of a lead corresponding to an acquisition source of the bio-signal to a mutually matched feature by using the neural network model. For example, the computing device 100 may include the lead-wise positional information in the feature of the lead-wise bio-signal generated through step S120. In this case, the lead-wise positional information may be reflected to the feature through a neural network included in the neural network model and may be reflected to the feature through a predetermined encoding scheme of converting the feature into a specific numerical code.

In step S140, the computing device 100 may derive the analysis value to which the correlation between the plurality of leads is reflected based on the feature encoded with the positional information of the lead through the neural network model. For example, even when not all lead-wise bio-signals corresponding to the plurality of leads but some lead-wise bio-signals corresponding to some combinations of the plurality of leads are received through step S110, the computing device 100 may derive a desired analysis value by reflecting the correlation between the plurality of leads through the neural network model. In this case, the neural network model may perform a self-attention based computation as a computation for reflecting the correlation between the plurality of leads. Further, in the self-attention based computation process, the neural network model may generate the matrix based on the feature encoded with the positional information of the lead in order to create a correlation based on the position between the plurality of leads.

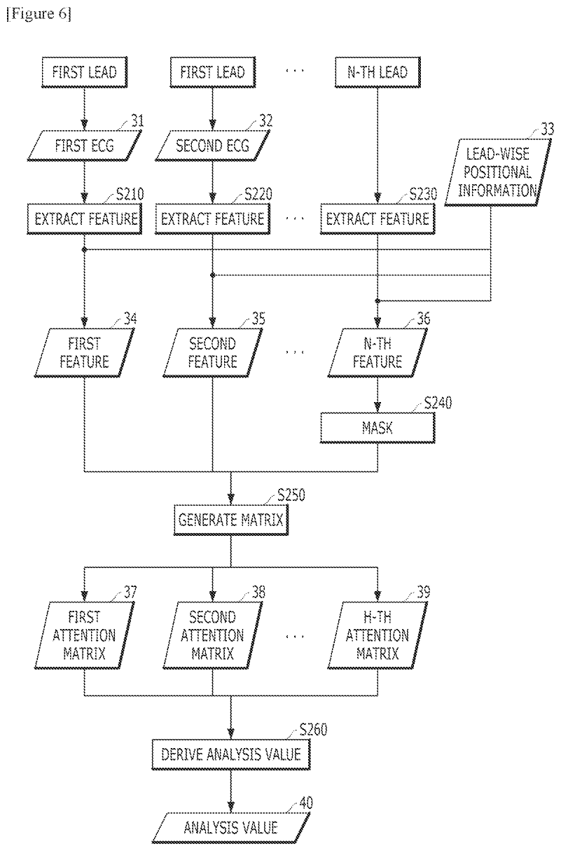

FIG. 6 is a flowchart illustrating a method for analyzing an ECG signal according to an embodiment of the present disclosure. Hereinafter, a situation in which an ECG signal is acquired from not all but some of N leads as one of the bio-signals according to an embodiment of the present disclosure is assumed.

The computing device 100 according to an embodiment of the present disclosure may perform operations S210, S220, and S230 of extracting a feature for the ECG signal acquired for each lead and an operation of encoding lead-wise positional information by using a neural network model. However, when a first ECG signal 31 is acquired from a first lead, a second ECG signal 32 is acquired from a second lead, and the ECG signal is not acquired from an N-th lead as illustrated in FIG. 6, the computing device 100 does not actually perform the feature extracting operation and the operation of encoding the lead-wise positional information corresponding to S230 because there is no data corresponding to the N-th lead. Therefore, unlike a first feature 34 and a second feature 35, an N-th feature 36 may be appreciated as blank data where there is no data value.

The computing device 100 may perform a masking operation (S240) for the N-th feature 36 which is the blank data so as for the neural network model to derive the analysis value by reflecting a correlation between N leads only by some ECG signals 31 and 32. Through the masking operation S240, the computing device 100 may generate a matrix value corresponding to the N-th lead among matrix values of a matrix to be generated through step S250 by using the neural network model (S240). The computing device 100 may generate the matrix based on the feature for the ECG signal acquired for each lead and the masked feature through the neural network model (S250). That is, the computing device 100 may generate an attention matrix through multi-heads of the neural network model based on the feature for the ECG signal acquired for each lead and the feature which is not acquired from the lead, and is masked. The computing device 100 may generate a first attention matrix 37, a second attention matrix 38, and an H-th attention matrix 39 according to the number of H heads based on the first feature 34 to which the positional information of the first lead is reflected, the feature 35 to which the positional information of the second lead is reflected, and the masked N-th feature. In this case, H may be a natural number of 1 or more. Meanwhile, in FIG. 6, it is expressed that the masking operation S240 precedes the matrix generation process S250, but the masking operation may be included in the matrix generation process S250.

The computing device 100 may derive an analysis value 40 for determining and/or predicting the health condition or disease of the subject based on the first attention matrix 37, the second attention matrix 38, and the H-th attention matrix 39 by using the neural network model (S260). For example, the analysis value 40 may include a probability value for determining and/or predicting a cardiovascular disease (e.g., myocardial infarction) of the subject for which ECG signal is measured.

In the meantime, according to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

Throughout the present specification, a calculation model, a nerve network, the network function, and the neural network may be used with the same meaning. Hereinafter, the terms of the calculation model, the nerve network, the network function, and the neural network are unified and described with a neural network. The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes.” The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

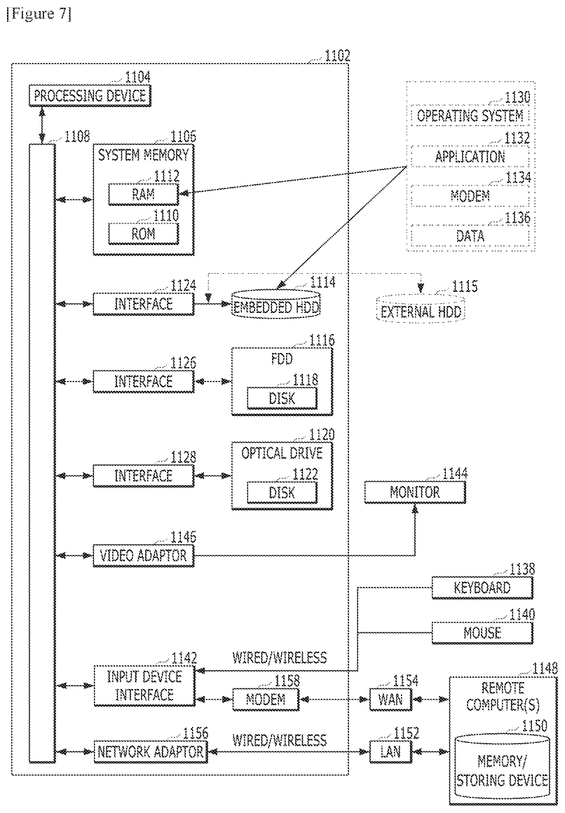

FIG. 7 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An exemplary environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined processor among various commercial processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))—the embedded HDD 1114 being configured for exterior mounted usage within a proper chassis (not illustrated)—a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

### Mode for Disclosure

Related contents in the best mode for carrying out the present disclosure are described as above.

### INDUSTRIAL APPLICABILITY

The present disclosure can be used in the computing device that analyzes the bio-signal.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A bio-signal analysis method performed by a computing device including at least one processor, comprising:
acquiring at least one lead-wise bio-signal from a plurality of leads; and
deriving an analysis value by inputting the acquired at least one lead-wise bio-signal into a neural network model,
wherein the analysis value is derived by reflecting a correlation between the plurality of leads based on the input at least one lead-wise bio-signal regardless of a combination of lead-wise bio-signals which are enabled to be acquired from the plurality of leads.

2. The method of claim 1, wherein the deriving of the analysis value includes:
extracting a feature for the acquired at least one lead-wise bio-signal by using the neural network model,
encoding positional information of a lead in which the bio-signal is acquired to the extracted feature by using the neural network model, and
deriving the analysis value to which the correlation between the plurality of leads is reflected based on the feature encoded with the positional information of the lead by using the neural network model.

3. The method of claim 2, wherein the deriving of the analysis value to which the correlation between the plurality of leads is reflected based on the feature encoded with the positional information of the lead includes:
performing a self-attention based computation for reflecting the correlation between the plurality of leads based on the feature encoded with the positional information of the lead by using the neural network model, and
deriving the analysis value based on a result of the self-attention based computation by using the neural network model.

4. The method of claim 3, wherein the performing of the self-attention based computation for reflecting the correlation between the plurality of leads includes:
generating a matrix for representing the correlation between the plurality of leads based on the feature encoded with the positional information of the lead by using the neural network model, and
deriving the result of the self-attention based computation based on the matrix by using the neural network model.

5. The method of claim 4, wherein the generating of the matrix for representing the correlation between the plurality of leads based on the feature encoded with the positional information of the lead includes:
generating a query vector, a key vector, and a value vector based on the feature encoded with the positional information of the lead by using the neural network model, and
generating a multi-head matrix based on the query vector and the key vector by using the neural network model.

6. The method of claim 5, wherein the deriving of the result of the self-attention based computation based on the matrix includes:
deriving a weighted sum of the value vector based on the multi-head matrix by using the neural network model.

7. The method of claim 5, wherein the generating of the multi-head matrix based on the query vector and the key vector includes:
masking a matrix value corresponding to a lead in which the bio-signal is not acquired in the multi-head matrix.

8. The method of claim 7, wherein the masking is to process the matrix value of the multi-head matrix as 0.

9. The method of claim 1, wherein the neural network model is pre-trained by randomly masking the matrix for representing the correlation between the plurality of leads and generated based on lead-wise bio-signals acquired in all of the plurality of leads.

10. A computer program stored in a computer-readable storage medium, wherein the computer program executes the following operations for analyzing a bio-signal when the computer program is executed by one or more processors, the operations comprising:
an operation of acquiring at least one lead-wise bio-signal from a plurality of leads; and
an operation of deriving an analysis value by inputting the acquired at least one lead-wise bio-signal into a neural network model,
wherein the analysis value is derived by reflecting a correlation between the plurality of leads based on the input at least one lead-wise bio-signal regardless of a combination of lead-wise bio-signals which are enabled to be acquired from the plurality of leads.

11. A computing device analyzing a bio-signal, comprising:
a processor including at least one core;
a memory including program codes executable in the processor; and
a network unit acquiring at least one lead-wise bio-signal from a plurality of leads,
wherein the processor derives an analysis value by inputting the acquired at least one lead-wise bio-signal into a neural network model, and
wherein the analysis value is derived by reflecting a correlation between the plurality of leads based on the input at least one lead-wise bio-signal regardless of a combination of lead-wise bio-signals which are enabled to be acquired from the plurality of leads.
